(19) United States
(12) Patent Application Publication
(10) Pub. No.: US 2023/0170767 A1
(43) Pub. Date: Jun. 1, 2023

(54) ROTO-LINEAR AXIS SYSTEM FOR PROBE POSITIONING

(71) Applicant: Xerxes Maximilian Libsch, White Plains, NY (US)

(72) Inventor: Xerxes Maximilian Libsch, White Plains, NY (US)

(21) Appl. No.: 17/922,166

(22) PCT Filed: Apr. 28, 2021
(86) PCT No.: PCT/US2021/029750

§ 371 Date: Oct. 28, 2022

Related U.S. Application Data
(60) Provisional application No. 63/016,438, filed on Apr. 28, 2020.

(51) Int. Cl. H02K 11/21; A61M 5/20; A61M 5/46

(52) U.S. Cl. CPC H02K 11/21; A61M 5/20; A61M 5/46; H02K 2213/03

(57) ABSTRACT

The disclosed system allows for the positioning of a sensor probe using a roto-linear mechanism. Specifically, the device allows for independent positioning of the probe along a linear axis and a rotary axis such that the linear axis includes a coupling that allows for arbitrary positioning of the linear axis with respect to the rotary axis. The probe can contain a plurality of sensors, such that the roto-linear mechanism can position the probe. In addition, the device allows for modular radial attachment points such that the probe can be radially positioned at arbitrary radii. The disclosed invention can be included in a system that includes software to manipulate and control the roto-linear motion of the device.

### CROSS-REFERENCE TO RELATED APPLICATIONS

The present application is the nationalization of PCT Application PCT/US2021/029750, filed Apr. 28, 2021, published as WO2021222473A1 on Nov. 11, 2021, said application incorporated herein by reference in its entirety. PCT/US2021/029750 is an international PCT application of and claims the benefit of U.S. Provisional Application No. 63/016,438, filed Apr. 28, 2020, which is incorporated herein by reference in its entirety.

### BACKGROUND OF THE INVENTION

There are several prior art devices.

Roto-Linear Actuator EP2790306A2. Disclosed systems and methods allow for roto-linear actuator that comprises a linear movement unit, translation shaft unit, and joint interposed between the movement units for engaging the rotation shaft with the translation shaft.

Rotolinear Actuator with Satellite Rollers WO2009081034A2. Disclosed systems and methods allow for a rotolinear actuator that comprises a central rod with an outer thread surrounded by a ring with an inner thread and containing threaded satellite rollers that are distributed between the central rod and the outer ring.

Electric Jack Having a Rotation-Translation Movement Conversion System and With Energy Recovery U.S. Pat. No. 5,444,348A. The invention disclosed contains an electric jack with a translation movement and at least on electric motor as well as means for controlling the motor.

Automatic Intravenous Injection Device EP2790306A2. Disclosed systems and methods allow for an intravenous injection device with specific claims as to fixed rings, elastic bands, handles and injector plates.

Vaccine Injection Device CN201631815U. Disclosed systems and methods allow for a needle piston and a core.

In general, a wide array of applications benefit from being able to precisely position a probe along a given surface. For example, in the field of manufacturing, manufacturing robots typically must precisely position probes containing welding tips, cutting tips or other system along a surface or substrate that is being manufactured. In addition, additive manufacturing techniques require precise positioning of probes along a surface for processes such as 3D printing.

In addition, the precise positioning of a probe along a given surface is beneficial to the field of automated tattoo machines, which require the positioning of a needle along a surface of tattoo application and into subcutaneous tissue in order to deliver the tattoo ink in order to construct the tattoo.

In addition, such precise positioning of a probe along a given surface is uniquely beneficial to the field of medical devices. Such devices could include needle injection devices which are used to inject a needle into a vein or artery in order to perform a variety of functions. Such cases can uniquely benefit from a roto-linear actuation system where the actuation allows for traversal across the injection plane in order to locate a suitable vein, and then normal to the injection plane in order to insert a probe such as a needle into the given vein.

In US Patent No. CN203303445U, an intravenous injection is presented that includes fixed rings, elastic bands, handles and injector plates. However, the invention does not allow for rotary positioning or any linear positioning of a given needle.

In US Patent No. CN201912553U, a similar intravenous injection device is presented that includes mechanisms for syringes and arm clamps. However, the invention does not contain movable axes that allow for rotational and linear motion.

In US patent No. systems and methods for autonomous intravenous are disclosed. However, the device encompasses a robot arm and a medical device pivotally attached to the robot arm. The actuation does not comprise roto-linear type actuation.

In US Patent No. EP2790306A2, a roto-linear actuator is described that comprises a linear movement unit, translation shaft unit and joint interposed between the movement units for engaging the rotation shaft with the translation shaft. The device also requires a joint between the rotational and linear axes and the units are connected in series with one another, and the device requires a bearing.

Despite prior approaches, there still exists a great need for a device, system and methodology that precisely positions a probe in a roto-linear fashion. The systems described have several major limitations that are not addressed in the prior art. First, such inventions regarding medical devices do not allow for the positioning of a probe in a roto-linear fashion where the axis of rotation is not collinear with the linear axis. Second, such inventions do not allow for the radius of the probe on the rotational axis to be adjusted, so that greater or lesser radii of translation can be achieved. Third, the inventions described performing roto-linear actuation require couplings and joints in order to engage the rotation shaft with the translation shaft and that do not provide arbitrary alignment of the linear and rotational axes.

The ultimate goal of this invention is to address the fundamental limitations aforementioned.

### SUMMARY OF THE INVENTION

The disclosed system allows for the positioning of a sensor probe using a roto-linear mechanism. Specifically, the device allows for independent positioning of the probe along a linear axis and a rotary axis such that the linear axis includes a coupling that allows for arbitrary positioning of the linear axis with respect to the rotary axis. The probe can contain a plurality of sensors, such that the roto-linear mechanism can position the probe. In addition, the device allows for modular radial attachment points such that the probe can be radially positioned at arbitrary radii. The disclosed invention can be included in a system that includes software to manipulate and control the roto-linear motion of the device.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1: is a schematic of the device according to an embodiment of the present invention, for roto-linear actuation for sensor probe positioning.

FIG. 2: is a schematic of the system according to an embodiment of the present invention, for roto-linear actuation for sensor probe positioning.

FIG. 3a: is a schematic of the methodology according to an embodiment of the present invention, for roto-linear actuation for sensor probe positioning.

FIG. 3b: is a schematic of the methodology according to an embodiment of the present invention, for roto-linear actuation for sensor probe positioning.

FIG. 3c: is a schematic of the methodology according to an embodiment of the present invention, for roto-linear actuation for sensor probe positioning.

FIG. 4: is a diagram showing a comparison of the roto-linear coordinate system with respect to a substrate and a traditional cartesian coordinate system.

FIG. 5: is a diagram showing the methodology for probe positioning with respect to a substrate.

FIG. 6: is a diagram showing another methodology for probe positioning with respect to a substrate.

### DETAILED DESCRIPTION OF THE INVENTION

### Device—Embodiment 1

The ultimate goal of this invention is to address the fundamental limitations aforementioned. One or more embodiments of the invention address these and other needs by providing a fundamentally different approach for sensor probe positioning utilizing a roto linear mechanism. The embodiment EMBODIMENT 1 shows a base modular mounting structure having dimensions from 1 mm2 to 10,000 m2 that allows a force measuring sensor to be rigidly coupled to it. The mounting structure is modular, such that one or more hardware units can be connected to it and contains a force sensor that is coupled to the structure. The force sensor is in turn coupled to a hollow probe suitable for liquid flow through the channel of the probe, directly in line with the axis of the force sensor. The device allows for arbitrary positioning of the force sensor and probe along the platform, such that the radius from the center of the platform can be controlled. The platform is connected rigidly to a rotary axis, which is driven by a motor. The motor is fixed to a coupling that allows for angular positioning of the motor and thus the platform with respect to a common reference plane normal to the probe tip. The coupling also attaches the motor to a linear rail carriage, which travels along a linear rail guide. The linear rail guide thus allows for the base modular mounting structure to move along a linear as well as rotary axis. The linear rail carriage is driven by a screw system that translates rotary motion from a second motor into linear motion along the axis of the linear rail guide. The second motor is attached to a base plate by means of another mounting bracket.

### System—Embodiment 2

One embodiment of the present invention is shown in EMBODIMENT 2. The device of EMBODIMENT 1 is augmented to include a base housing. The base housing in addition houses a microcontroller. Upon the base housing rests a roto-linear axis system to position a base modular mounting structure in along a rotational and linear motion profile. This is achieved by a linear rail guide system wherein the output of one motor shaft drives a lead screw, which then translates the rotary motion to linear motion of the linear rail guide. This, in turn, drives a linear rail carriage along the linear rail guide. A rotary motor is mounted to the linear rail guide via a mounting bracket that allows for arbitrary rotation of the motor parallel to the ground plane. A base modular mounting structure is coupled to the output shaft of the rotary motor, which allows for the rotational movement of the base modular mounting structure around the axis of the motor. In addition, the base modular mounting structure contains a probe that is hollow in nature to allow fluid to pass through, and can be positioned at any arbitrary point radially along the base mounting structure. Ultimately, the system allows for roto-linear positioning of the base modular mounting structure.

### Process—Embodiment 3a

Another embodiment of the present invention relates to the process by which encoding sensors may be used in order to determine the position or trajectory of each axis relating to rotolinear motion. At each time or motion step for the system in EMBODIMENT 2, sensor output from the encoding sensor along each output is used. A difference between the current step and previous step for the roto-linear device from EMBODIMENT 1 is used in order to obtain the current position of each axis.

### Process—Embodiment 3b

In addition, the embodiment of the present invention relates a methodology that may utilize an algorithm, and a force sensor to position the probe to an arbitrary position using roto linear motion to the surface of a substrate. For each encoding sensor, local positioning of each axis is obtained by comparing encoding sensor differentials at each point along the motion path. In addition, force sensor output is recorded, such that upon contact of the probe from EMBODIMENT 1 with the substrate, the probe transfers a force to the force sensor sufficiently attached to the modular platform. Using this force sensor data in addition to the encoding sensors thus allows for the position of the substrate to be found with respect to the axes of roto-linear motion.

### Process—Embodiment 3c

In addition, the embodiment of the present invention relates to a methodology that may utilize an algorithm as well as pre-tabulated force sensor data in order to determine position of the probe within a substrate using the force sensor. Using pre-tabulated data of known force outputs upon insertion of a probe into a substrate, and comparing the known signal with the force sensor output allows for a mapping between force signal and position within the substrate to be utilized in order to determine the position of the probe within a particular substrate.

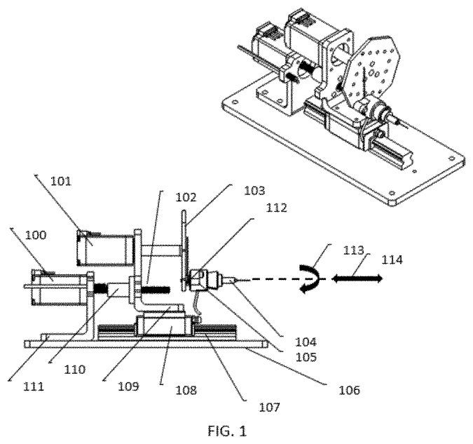

A further description of the example embodiments of the invention follows. Embodiments of the claimed invention can be first explained with reference to FIG. 1.

FIG. 1 is a schematic of the device according to an embodiment of the present invention. The device contains a base modular mounting structure (103) with dimensions 1 mm2 to 10,000 m2 that is functionally connected to at least one or more other hardware units. This includes a force sensor (112), which can be capable of measuring in the range 1 mN to 10,000 N. This force sensor is coupled to the modular mounting structure and contains a magnetically coupled probe. The force sensor is mounted directly to the base modular mounting structure by means a threaded screw system. The force sensor contains a thread screw system on the positive end (outward facing), to which a magnetic fixture is tapped and attached (105). The magnetic fixture allows for a magnetic collet attached to a probe (104) that contains a channel for the flow of liquids to flow through to be attached non-permanently. This allows for repeatable and accurate alignment that does not induce bias to force sensing signals that would have otherwise resulted from rigid attachment. The modular mounting platform is in turn attached to the output shaft of a motor (101) which allows it to turn in a rotary fashion along a central axis (113). This motor is then coupled to a mechanical fixture (109) which holds it rigidly to a linear carriage (108) by means of screw based attachment. The linear carriage is free to slide along a linear rail (107), which is itself mounted to a baseplate (106) which supports the entire device. The linear rail is driven by a lead screw and nut system (110), which converts rotary motion from the output shaft of another motor (100) into linear motion along a given linear axis (114). This motor is in turn attached to the baseplate by means of a mechanical fixture (111).

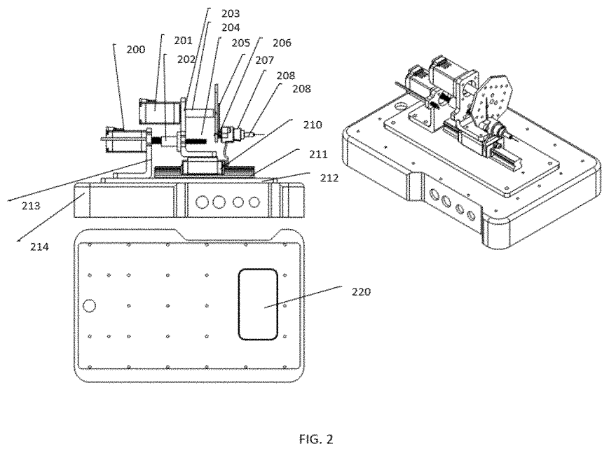

A further description of the invention can be explained with reference to FIG. 2.

In FIG. 2, the device from FIG. 1, including the motors (200, 201), screw and nut drive (202), mechanical fixtures to hold the motors (203, 213), linear rail and carriage (210, 211), modular mounting structure (205), force sensor (206), magnetic fixture (207) and magnetic collet around a hollow probe (207, 208) is attached to a base mounting structure (214). This base mounting structure houses a microcontroller (220).

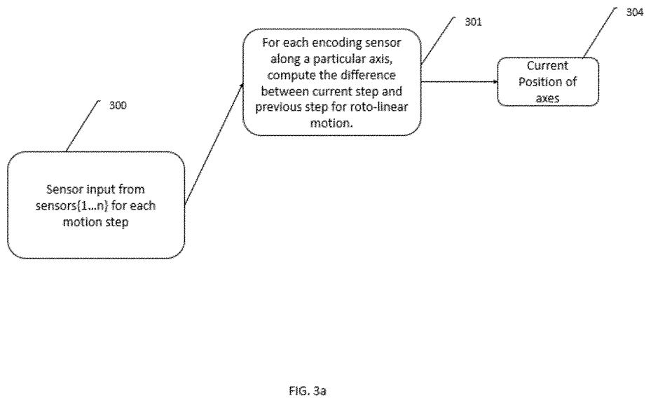

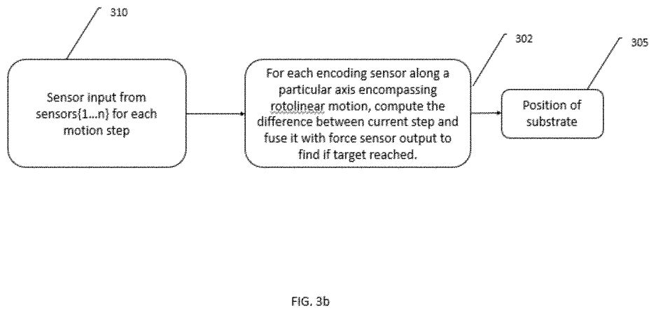

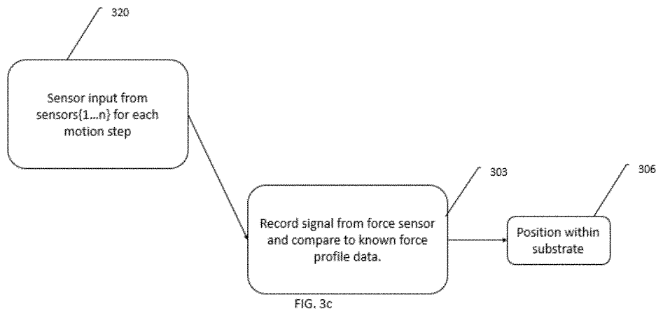

A further description of the invention can be explained with reference to FIG. 3a.

FIG. 3a is a schematic of the system according to an embodiment of the present invention showing the methodology by which the current position of each axis of motion relating to EMBODIMENT 1 (304). Sensor input from each sensor, including force sensors and encoding sensors is collected for each motion step or time step (300). For each encoding sensor along a particular axis, the difference between current step and previous step is computed in order to generate the current position of each axis (301).

A further description of the invention can be explained with reference to FIG. 3b.

FIG. 3b is a schematic of the system according to an embodiment of the present invention showing the methodology by which position of the substrate with respect to each axis in EMBODIMENT 1 can be found (305). Sensor input from each sensor, including force sensors and encoding sensors is collected for each motion step or time step (310). In addition, for each encoding sensor along a particular axis encompassing roto-linear motion, the encoding sensor data is compared with force sensor data, such that when the probe sufficiently attached to the force sensor data nad modular platform touches a particular substrate, the force sensor data registers this change in force resulting from the contact with the substrate, and the position of the substrate is thus found (302).

A further description of the invention can be explained with reference to FIG. 3c.

FIG. 3c is a schematic of the system according to an embodiment of the present invention showing the methodology by which the position of the probe with respect to EMBODIMENT 1 can be found (306). Sensor input from each sensor, including force sensors and encoding sensors is collected for each motion step or time step (320). In addition, the force sensor signal is recorded while the probe is inserted into the substrate, such that the force data can be compared with known force sensor data and thus a position within the substrate can be found through the force position mapping. Note that those skilled in the art would recognize that one or more methodologies encompassing, but not limited to those described in FIG. 3a, FIG. 3b, and FIG. 3c can be used simultaneously or independently. Such methodologies can also be implemented with feedback to each other, and that other feedback methodologies may be incorporated.

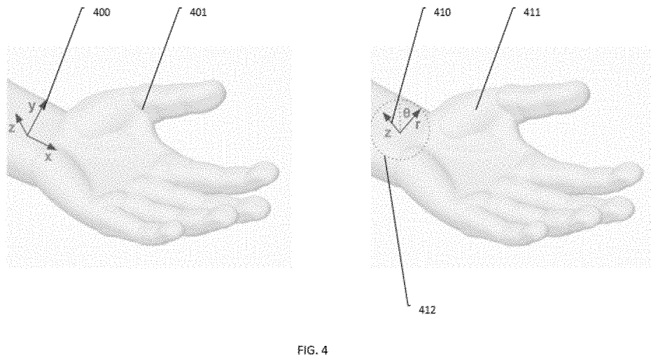

A further description of the invention can be explained with reference to FIG. 4.

FIG. 4 shows a comparison between a typical cartesian coordinate system with 3 mutually perpendicular axes (400) and the roto-linear coordinate system (410) with respect to a human forearm (401, 411). The roto-linear axis positioning system from EMBODIMENT 1 positions radially using the rotational system according to rotation in plane with the forearm (referred to as a theta rotation), radially outward from an arbitrary point on the forearm (referred to as r) by arbitrarily changing the distance along the modular base platform of the functionally coupled probe, and perpendicular to the forearm (referred to as z) by utilizing the linear rail system driven by another motor. The radial path of motion sweeps out a circular path with respect to the plane of the forearm (412).

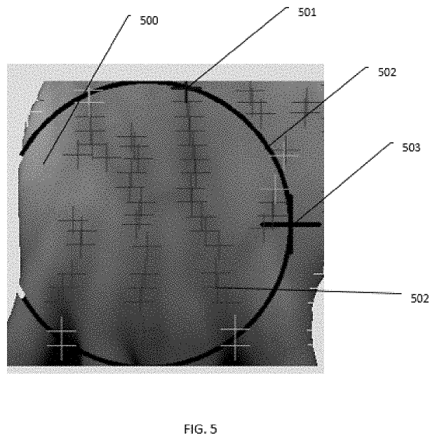

A further description of the invention can be explained with reference to FIG. 5.

FIG. 5 shows a methodology that may utilize an algorithm and encoding sensors to position the probe from EMBODIMENT 1 to an arbitrary position using roto linear motion. A camera sensor is used to take an image of a hand (500). An algorithm is then used to highlight target probe points (502 representative), and project axes of motion onto a substrate. The current projected position of the probe onto the hand plane is represented as (503). The roto-linear mechanism can translate the probe in a rotary fashion on the hand plane (502), which can be used to precisely position the probe to a specified location (501).

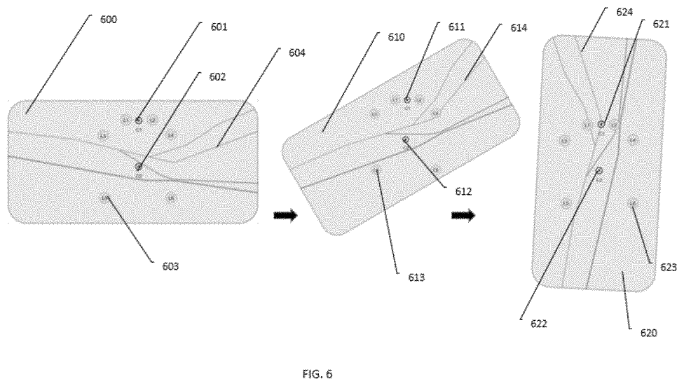

A further description of the invention can be explained with reference to FIG. 6.

FIG. 6 shows the same substrate of a human forearm during three phases of the roto-linear mechanism action (600, 610, 620). A target vein (604) is chosen to which to position the probe from EMBODIMENT 1. The centroid of the probe is projected onto the substrate (601), as well as the center of the based modular mounting structure (602). In addition, other arbitrary projection points from the base modular mounting structure (603 representative) are shown. The system from EMBODIMENT 2 positions the device rotationally in order to achieve a rotation with respect to the substrate. This is shown by the rotated substrate (610), target vein (614), probe projection (611), modular mounting structure centroid projection (612), and other arbitrary projection points from the base modular mounting structure (613). The end position of the rotational motion yields a positioning of the probe directly above the target point, such that the system from EMBODIMENT 2 can insert the probe into the target position along the substrate by employing linear motion of the system. This is shown by the rotated substrate (620), target vein (624), probe projection (621), modular mounting structure centroid projection (622), and other arbitrary projection points from the base modular mounting structure (623).

While this invention has been particularly shown and described with reference to example embodiments thereof, it will be understood by those skilled in the art that various changes in form and details may be made therein without departing from the scope of the invention encompassed by the appended claims.

All references cited herein are incorporated herein by reference to the full extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference.

What is claimed is:

1. A positioning device comprising:
at least on rotary axis and at least one linear axis;
a base modular mounting structure;
a probe that is functionally coupled to the base modular mounting structure; and a coupling between each axis that allows for arbitrary mechanical alignment between axes.

2. The device according to claim 1, wherein the rotary axes are free to rotate from 0 degrees to 360 degrees and the linear axes are free to move in the range of 1 nm minimum to 100 m maximum.

3. The device according to claim 1, wherein the base modular mounting structure has surface dimensions from about 0.1 mm² to 100 m².

4. The device according to claim 1, wherein the base modular mounting structure allows for functional coupling of the probe at any arbitrary radius from the center of the modular mounting structure.

5. The device according to claim 1 wherein the probe may contain a plurality of sensors.

6. The device according to claim 1, wherein the probe contains a channel for the flow of liquids to flow in either direction along the channel.

7. The device according to claim 1 wherein the all possible position targets of the probe along a surface can be reached with a rotary (theta) and linear (z) transformation about and normal to the surface respectively, relative to the current position of the probe.

8. A positioning system comprising:
at least on rotary axis and at least one linear axis;
a base modular mounting structure;
a probe that is functionally coupled to the base modular mounting structure;
a coupling between each axis that allows for arbitrary mechanical alignment; and
allowances for containing feedback between the probe and each axis of motion.

9. The system according to claim 8, wherein the rotary axes are free to rotate from 0 degrees to 360 degrees and the linear axes are free to move in the range of 1 nm minimum to 100 m maximum.

10. The system according to claim 8, wherein the base modular mounting structure has surface dimensions from about 0.1 mm² to 100 m².

11. The system according to claim 8 wherein the probe can contain a plurality of sensors.

12. The system according to claim 8, wherein the system includes a microcontroller or similar means of handling input, output and data processing.

13. The system according to claim 8, wherein the microcontroller might include memory, software and algorithms.

14. The system according to claim 8, wherein the feedback can be controlled using software and algorithms.

15. A method of positioning comprising:
at least on rotary axis and at least one linear axis;
a base modular mounting structure;
a probe that is functionally coupled to the base modular mounting structure; and a coupling between each axis that allows for arbitrary mechanical alignment; and
a methodology that at least uses encoding sensors to determine the position or trajectory of each axis.

16. The methodology according to claim 15 wherein the methodology is iterative based on a time frame between 1 nanosecond and 1 hour.

17. A method of positioning comprising:
at least on rotary axis and at least one linear axis;
a base modular mounting structure;
a probe that is functionally coupled to the base modular mounting structure; and a coupling between each axis that allows for arbitrary mechanical alignment; and
a methodology that at least uses an algorithm, and a force sensor to position the probe to an arbitrary position using roto linear motion to the surface of a substrate.

18. The methodology according to claim 17 wherein the methodology is iterative based on a time frame between 1 nanosecond and 1 hour.

19. A method of positioning comprising:
at least on rotary axis and at least one linear axis;
a base modular mounting structure;
a probe that is functionally coupled to the base modular mounting structure; and a coupling between each axis that allows for arbitrary mechanical alignment; and
a methodology that may at least use an algorithm as well as pre-tabulated force sensor data in order to determine position of the probe within a substrate using the force sensor.

20. The methodology according to claim 19 wherein the methodology is iterative based on a time frame between 1 nanosecond and 1 hour.
